(19) United States
(12) Patent Application Publication
(10) Pub. No.: US 2011/0081672 A1
(43) Pub. Date: Apr. 7, 2011

(54) PROCESS CONTROL OF BIOTECHNOLOGICAL PROCESSES

(76) Inventors: Steen Kjaer Andersen, Krogstens Alle (DK); Johan Weimann, Atoften (DK); Anders Broe Bendtsen, Bogholmen (DK)

(21) Appl. No.: 12/736,971

(22) PCT Filed: Jun. 13, 2008
(86) PCT No.: PCT/EP2008/057480

§ 371 Date: Nov. 29, 2010

(51) Int. Cl. C12Q 1/40; C12P 7/02; C12Q 1/00; C12Q 1/02; C12Q 1/34; C12M 1/36

(52) U.S. Cl. 435/22; 435/155; 435/4; 435/29; 435/18; 435/286.1

(57) ABSTRACT

A biotechnological process for conversion of a raw material (100, 200) to a desired product (130, 230) by means of one or more biological or biochemical agents (102, 104, 202) such as microorganisms and/or enzymes characterised in that the amount of one or more of said biological or biochemical agents (102, 104, 202) is controllable by a process control algorithm (124,224) dependent on one or more values of interest related to a process stream. A specific aspect of the invention is the use of a process control algorithm for controlling enzyme addition in biofuel production by fermentation of biomass to alcohols.

The invention is related to the technical field of process control in relation to a biotechnological process.

As the concern over greenhouse gas emissions increases, the production of so called biofuels be-comes increasingly important. Alcoholic biofuels may be produced directly by a biological process, which commonly is yeast fermentation of sugars, such as the sugars found in sugar canes and sugar beets. The biological process may also use other microorganisms such as bacteria to consume a carbohydrate feed to produce alcohol; most often ethanol, but also methanol and butanol are common examples of socalled bioalcohol fuels. Other raw materials such as grain and straw may also contain higher carbohydrates such as starch and/or cellulose, but in this case the starch and cellulose must be converted to sugars by an enzymatic process. The use of these two complex carbohydrates does however differ in that amylase enzymes for hydrolysis of starch are currently commercially available for this purpose in the so called first generation processes, whereas cellulase enzymes for hydrolysis of cellulose in the so called second generation processes have not gained wide usage yet.

Both the first and second generation processes, producing alcohols from starch and cellulose respectively, has two overall process steps; One or more initial enzymatic process steps are converting starch or cellulose to sugars available for fermentation and a subsequent fermentation is generating alcohol from sugars. While the initial enzymatic process steps releasing sugars from cellulose appear as two separate enzymatic reactions, the process equipment may still be designed for this part of the process to take place in a single reactor or in separate reactor. The fermentation is most often in a separate reactor, but may also take place in a single reactor.

As the enzymes for converting biomass to fermentable sugars constitute a significant portion of the cost of running a bio-ethanol production, in the socalled liquification (starch to polysaccharide conversion by amylase enzymes) and saccharification (polysaccharide to fermentable sugars conversion by gluco-amylase enzymes) and the effective use of enzymes is an important focus area. For this reason a great effort is made to identify the optimum temperature, pH and other operational conditions of the liquification and saccharification process for specific bio-mass sources, and accordingly the operation of a bio-ethanol plant is characterised by a high level of process monitoring.

Process control of industrial fermentation processes is often based on monitoring of the composition of the feed and effluent flows of a fermentor, and the rate of fermentation. Based on this information the composition of the feed, the fermentor temperature etc. is controlled, especially with focus on avoiding excess oxygen which will result in acetate formation, while maintaining the highest possible rate of reaction.

The practices of process operation are based on the experiences from biotechnological production of enzymes and pharmaceuticals as well as the production of wine and beer. For these processes the composition and quality of raw materials is fairly well defined, and the value of the end products is typically very high, and accordingly a high probability of successful production becomes more valuable than savings on the biological and biochemical agents and raw materials used in the process, and accordingly the recipes of operation may often define the use of excess supporting biological and biochemical agents such as enzymes and microorganisms.

However in the case of biofuel production the economic value of the product is lower compared to pharmaceuticals and at the same time the variation in composition and structure of the raw materials will often be significantly higher. The consequence of this is that the relative importance of the supporting biological and biochemical agents becomes more important, both from a technical perspective and from a economic perspective.

In e.g. the food industry, knowledge of the varying composition of a process feed of natural materials is important for process operation, e.g. for standardising a variable fat content of raw milk to the specified amount of fat in skimmed milk.

It is the objective of the present invention to make operation of biotechnological processes employing raw materials with a natural variation more robust and economically optimal.

The present invention combines the analysis of variable process streams with automated control of the addition of supporting biological and biochemical agents such as microorganisms and enzymes. As an example analysis of the feed of natural material in a bioethanol production process will reveal the varying amounts of readily available sugars, starch and cellulose. This detailed knowledge of the feed composition may be used in a feed forward control of the bioethanol production process parameters; including the key parameters of preparation processes including temperature and additions of supporting biological and biochemical agents, including amylases, cellulases and other enzymes.

Similarly analysis of the output or any other process stream from a biotechnological process employing a raw material with a natural variation may also be used in a feed-back control scheme to control the amounts of biological and biochemical agents added, or other important process parameters.

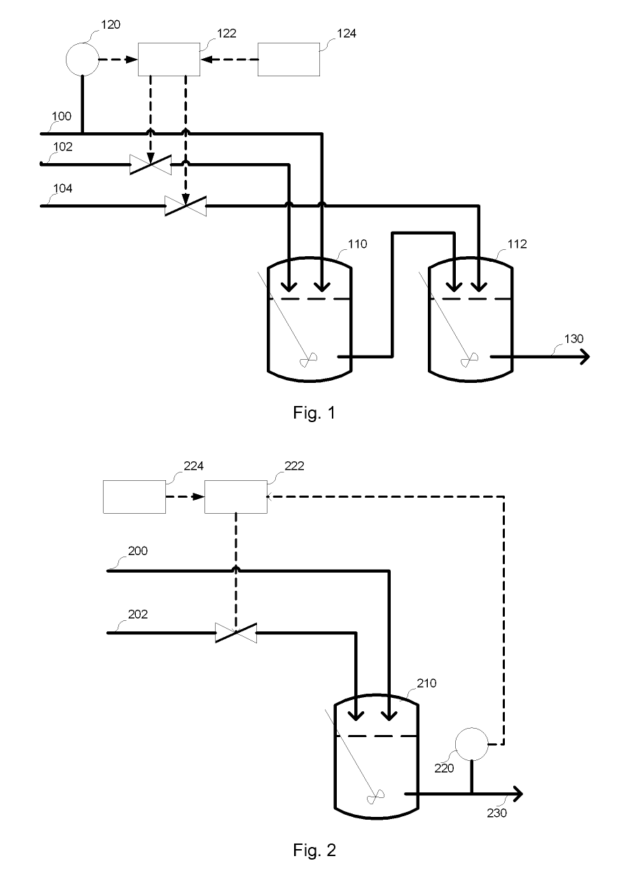

FIG. 1 shows conceptually a system of two bio-reactors in series, with feed forward control of enzyme addition from analysis of reactor inlet composition.

FIG. 2 conceptually shows a system with a single bio-reactor with feed back control of enzyme additions based on reactor outlet composition.

In FIG. 1 is shown an embodiment of the invention, in which a major feed stream of raw material for conversion in a biotechnological process 100 is led to a first reactor 110 and wherein a suitable first supporting biological and biochemical agent feed 102 to the first reactor 110 contains supporting biological and biochemical agents, such as microorganisms and enzymes suitable for a first biochemical preparation of the raw material. The major feed stream 100 is equipped with a suitable means of analysis 120, suitable for on-line or at-line use, such as a spectrometer employing absorption, transmission, reflection, attenuated total reflection, fluorescence, or Raman spectroscopy in combination with one or more signals related to electromagnetic radiation in one or more of the wavelength ranges, ultraviolet (200-400 nm), visible (400-700 nm), near-infrared (700 nm-2.5 μm), infrared (2.5-10 μm), far infrared (10-100 μm), terahertz (100 μm-1 mm) or microwave (1 mm-100 mm); or employing other types of analytical technology such as mass spectroscopy, ion mobility spectroscopy, nuclear magnetic resonance spectroscopy, gas chromatography, high performance liquid chromatography, capillary electrophoresis, bio-sensors, electrochemical sensors, and gas sensors, or determining a value of interest such as the concentration of constituents of interest in the raw material feed stream 100. The output of the means of analysis 120 is used as input to a to suitably configured data processing unit 122 consisting of one or more units, which may or may not be physically interconnected, which then based on a suitable control algorithm 124, such as but not limited to PID controllers, fuzzy logic control, simulation model based control, neural network based control, controls the amount of first supporting biological and biochemical agents 102 added. The outlet from the first reactor is led to a second reactor, together with a suitable second supporting biological and biochemical agent feed 104. The amount of this second supporting biological and biochemical agent 104 is also controlled by the second output 126 of the data processing unit 122 based on the composition of the raw material 100 as determined by the means of analysis 120.

The process thus controlled may be any biotechnological process, or any sub-process of an overall biotechnological process, but processes in which the raw material feed stream 100 contains or derives from a raw material of natural origin will benefit especially from process control based on concentrations of constituents, as determined by a means of analysis 120, due to the natural variation of raw materials. An example of this are processes producing ethanol or other alcohols as the product 130 from biomass raw materials 100 containing starch or cellulose, such as grain, maize, wood, algae, switch grass and other suitable biomass raw materials wherein the reaction in the first reactor 110 will be the enzymatic conversion of starch or cellulose into fermentable sugars by addition of a suitable amount of enzymes such as amylase or cellulase as the first supporting biological and biochemical agent feed 102, and the conversion in the second reactor 112 will be the fermentation of sugars into ethanol, with the aid of suitable yeast or bacteria as the second supporting biological and biochemical agent feed 104. FIG. 1 can also represent an intermediate step of such a fermentation process, where the raw material stream 100 is an intermediate outlet from the liquification process step.

In FIG. 2 is shown an alternative embodiment of the invention. In this embodiment a single reactor 210 is used, whereto a major feed stream of raw material 200 for consumption in a biotechnological process is led, and wherein a suitable supporting biological and biochemical agent feed 202 containing supporting biological and biochemical agents, such as microorganisms and enzymes, is led to the reactor 210. An outlet stream 230 from the reactor is then led to later steps in the process. A value of interest such as the concentration of constituents of interest in the outlet stream 230 from the reactor 210 is determined by a means of analysis 220. The means of analysis 220 may be any means of quantitative analysis suitable for on-line or at-line use, such as spectrometers employing absorption, transmission, reflection, attenuated total reflection, fluorescence, or Raman spectroscopy in combination with one or more signals related to electromagnetic radiation in one or more of the wavelength ranges, ultraviolet (200-400 nm), visible (400-700 nm), near-infrared (700 nm-2.5 μm), infrared (2.5-10 μm), far infrared (10-100 μm), terahertz (100 μm-1 mm) or microwave (1 mm-100 mm); or employing other types of analytical technology such as mass spectroscopy, ion mobility spectroscopy, nuclear magnetic resonance spectroscopy, gas chromatography, high performance liquid chromatography, capillary electrophoresis, bio-sensors, electrochemical sensors, and gas sensors. By using the output of the means of analysis as input to a to suitably configured data processing unit 222, which consists of one or more units, which may or may not be physically interconnected, which then, based on a suitable control algorithm 224 controls an amount of supporting biological and biochemical agents 202 added. The control algorithm 224 thus employed may be of any type, such as but not limited to PID controllers, fuzzy logic control, simulation model based control, neural network based control, but an algorithm involving an explicit or implicit determination of the rate of reaction, e.g. by calculating the changes of metabolite content as a function of time may be especially useful, since changes in the rate of reaction may indicate inhibition of the biotechnological process, and may be compensated by appropriate adjustment of the amount or composition of the supporting biological and biochemical agent 202 added.

The process thus controlled may be any biotechnological process, and as in the first embodiment, processes in which the major feed stream 202 contains a natural raw material, will especially benefit from the determination of a value of interest such as the concentration of constituents of interest by use of a means of analysis 220 in connection with a process control algorithm (224). Again an example of this may be processes producing ethanol as the product 230 from biomass raw materials 200 such as grain, maize, wood, algae, switch grass and other suitable biomass raw materials wherein the reaction in the reactor 210 will a combined enzymatic conversion of starch or cellulose into fermentable sugars and sugar to ethanol fermentation by addition of a suitable amount of enzymes such as amylase, gluco-amylase, alpha-amylase, and cellulase and microbiological organisms such as yeast or bacteria in the supporting biological and biochemical agent feed 202.

The person skilled in the art will realise that the processes and systems involving an intermediate step or an overall process in relation to bioalcohol production, will benefit from monitoring concentrations of constituents, including raw materials, intermediates, desired end products or undesired end products of the fermentation process, including monosaccharides, disaccharides, oligosaccharides and polysaccharides, as well as alcohols, organic acids, fermentation inhibitors and indicators of fermentation stress or fermentation infections, resulting in the following non-exhaustive list of constituents which may be of interest for process control; sugars, including monosaccharides; further including pentoses including arabinose, deoxyribose, lyxose, ribose, ribulose, xylose and xylulose and hexoses further including glucose, galactose, mannose, gulose, idose, talose, allose, altrose, fructose, sorbose, tagatose, psicose, fucose, fuculose, and rhamnose

and disaccharides including sucrose, lactose, trehalose, maltose and cellobiose
alcohols, such as methanol, ethanol, propanol and butanol; glycerol, organic acids, such as lactic acid, acetic acid, and succinic acid and higher carbohydrates, such as oligo-saccharides, such as DP3, DP4, DP3+ and DP4+, and fermentation inhibiting constituents such as hydroxymethylfurfural and furfural, and macromolecules such as starch, celluloses, lignocellulose and protein.

The means of analysis 120,220 described in the two embodiments is preferably a type which is suitable for on-line instrumentation, but it may also be an instrument positioned at-line. In the case of an at-line instrument a sample will be taken from the process to the instrument, and the parameter of interest may either be transmitted directly to the process control algorithm 124, 224 or entered manually to the data processing unit 122, 222.

As will be realised by the person skilled in the art, the embodiments presented are simplifications with focus on the present invention, to enhance the readers understanding of this invention. The omission of other controlled or monitored variables including temperature, pH, amount of nutrients, effluent gas composition, does not imply that such variables can not be part of a control scheme covered by the invention.

Similarly the person skilled in the art will realise that any biotechnological process may benefit from the invention, and not just the specific processes mentioned in the embodiment and the description. This will also include processes in which supporting agents are controlled and added in more individual streams, or where the process is operated in another reactor type, including but not limited to batch reactors and plug flow reactors.

The person skilled in the art will also realise that the practical implementation of a control scheme covered by the present invention may be based on other values of interest from the means of analysis or even the raw data or intermediate data from the means of analysis (120, 220) instead of the specifically mentioned one or more parameters of interest.

1. A biotechnological process for conversion of a raw material to a desired product by means of one or more biological or biochemical agents such as microorganisms and/or enzymes characterised in that the amount of one or more of said biological or biochemical agents is controllable by a process control algorithm dependent on one or more values of interest related to a process stream.

2. A biotechnological process according to claim 1, equipped with a means of analysis wherein the process control algorithm is configured to receive, as input, one or more values of interest related to said raw material as determined by a means of analysis.

3. A biotechnological process according to claim 1 for conversion of a raw material to a desired product by means of one or more biological or biochemical agents such as microorganisms and/or enzymes wherein the process control algorithm is configured to receive, as input, one or more values of interest related to the outlet of said process as determined by a means of analysis.

4. A biotechnological process according to claim 3 where the one or more values of interest related to the outlet of said process is taken from the group consisting of concentrations of raw materials or intermediates of said process or sub-process, desired products of said process or sub-process, undesired products of said process or sub-process and rate of reaction of said process or sub-process, as determined from the output of said means of analysis.

5. A biotechnological process according to claim 2 where said means of analysis is a device employing one or more of the following group of analytical technologies
spectroscopy employing transmission, reflection, attenuated total reflection, fluorescence, or raman spectroscopy in combination with one or more signals related to electromagnetic radiation in one or more of the wavelength ranges, ultraviolet (200-400 nm), visible (400-900 nm), near-infrared (900 nm-2.5 m), infrared (2.5-10 m), far infrared (10-100 m), terahertz (100 m-1 mm) or microwave (1 mm-100 mm); mass spectroscopy, ion mobility spectroscopy, nuclear magnetic resonance spectroscopy, gas chromatography, high performance liquid chromatography, capillary electrophoresis, bio-sensors, electrochemical sensors, and gas sensors.

6. A biotechnological process according to claim 1 where the biotechnological process is a process for production of an alcohol.

7. A biotechnological process according claim 1 where one or more of the values of interest are taken from the list consisting of pH and concentrations of constituents taken from the group consisting of sugars, including monosaccharides; further including pentoses including arabinose, deoxyribose, lyxose, ribose, ribulose, xylose and xylulose and hexoses further including glucose, galactose, mannose, gulose, idose, talose, allose, altrose, fructose, sorbose, tagatose, psicose, fucose, fuculose, and rhamnose and disaccharides including sucrose, lactose, trehalose, maltose and cellobiose
alcohols, such as methanol, ethanol, propanol and butanol glycerol,
organic acids, such as lactic acid, acetic acid, and succinic acid and higher carbohydrates, such as oligo-saccharides, such as DP3, DP4, DP3+ and DP4+,
fermentation inhibiting constituents such as hydroxymethylfurfural and furfural, and macromolecules such as starch, celluloses, lignocellulose and protein.

8. A biotechnological process according to claim 1 where one or more of the values of interest are indicators of one or more of the following; the degree of saccharide polymerisation, the fermentability of biomass, the microbiological status of fermentation, such as fermentation infections, microorganism stress, microorganism inhibition, and rate of fermentation.

9. A biotechnological process according to claim 2 where one or more of the values of interest are raw or intermediate data from the means of analysis.

10. A biotechnological process according to claim 1 where the one or more biochemical agents is taken from list comprising amylase, gluco-amylase, alpha-amylase, and cellulase.

11. A system for carrying out a biotechnological process comprising one or more reactors having an inlet of one or more biological or biochemical agents, a means of analysis, a data processing unit and a process control algorithm implemented in a means of process control mutually configured to produce a desired product by a process to claim 2.
